(19) United States
(12) Patent Application Publication
(10) Pub. No.: US 2014/0058293 A1
(43) Pub. Date: Feb. 27, 2014

(54) Multi-Frequency Ultrasound Device and Method of Operation

(71) Applicant: Sunnybrook Research Institute, Toronto (CA)

(72) Inventors: Kullervo Hynynen, Toronto (CA); Meaghan O'Reilly, North York (CA)

(73) Assignee: Sunnybrook Research Institute, Toronto (CA)

(21) Appl. No.: 13/901,228

(22) Filed: May 23, 2013

Related U.S. Application Data
(60) Provisional application No. 61/650,607, filed on May 23, 2012.

(51) Int. Cl. A61B 8/00; A61B 8/08; A61N 7/02

(52) U.S. Cl. CPC A61B 8/4488; A61N 7/02; A61B 8/481
USPC 601/2

(57) ABSTRACT

Apparatus and method for delivering increased amounts of energy to localized treatment zones at a target location are provided. In some instances, using gated pulses of ultrasound in a multi-frequency applicator, microbubbles are generated or excited in or near the target location, for example in a patient's tissue or blood stream for enhanced delivery of ultrasound energy to the patient. Applications include ablation of diseased tissue, thrombolysis, blood-brain barrier disruption or tissue diagnosis.

### RELATED APPLICATIONS

This application claims the benefit and priority of U.S. Provisional Application No. 61/650,607, filed on May 23, 2012, which is incorporated herein by reference.

### TECHNICAL FIELD

The present application is generally directed to the design and operation of acoustic systems. In some examples these may be used for the treatment of certain conditions in patients using an apparatus that provides focused ultrasonic energy to cause either therapy in a treatment volume of tissue in such patients, or diagnosis. More particularly, the invention relates to the excitation of gas bubbles in medical procedures using amplitude modulated multi-frequency transducer systems.

### BACKGROUND

Thermal therapy for diseased tissues and other conditions may be achieved through conversion of ultrasonic acoustic energy to thermal energy (heat) in or around the affected tissue or target site. The application of focused ultrasonic fields to a target zone or region of interest has been promising as it allows controlled and non-invasive heating of such regions by way of a focused or phased transducer source or array. The focal zone of such thermal therapy applicators can be in the few millimeter range, which allows heating of certain volumes of tissue without invasive surgical procedures. Such techniques also permit real time monitoring of the heated region by way of other imaging modalities such as magnetic resonance imaging (MRI).

Surgery using focused ultrasound beams has been carried out in animals and human patients for a variety of clinical conditions. Focused ultrasound surgery (FUS) has been used to treat human brain tumors, to perform spinal commissurotomy, and to treat glaucoma. Several clinical trials have used prototype ultrasound devices to treat benign and malignant tumors of prostate, bladder, and kidney. More recently several clinical trials using diagnostic ultrasound to guide the surgery have been reported with encouraging results. Existing systems generally rely on mechanical movement of a single focused transducer that produces a small focal volume resulting in long treatment times if the diseased region (e.g., tumor) has a substantial size to treat.

The potential for using phased arrays for ultrasound surgery has also been explored. To focus an acoustic beam, for therapy or diagnostic uses, an applicator is constructed from an array of small transducer elements, which are independently driven, or driven in discrete groups. An intensity maximum is created by driving the transducer elements in such a way that the waves emitted by each element are in phase at the desired focal point. The focusing is caused by superposition or constructive interference of the waves at the desired point, giving the ultrasonic field its highest intensity at the focus. Outside of the focal area the waves interfere more or less destructively or not coherently, thus minimizing the effect on the tissue the waves traverse prior to or after the focal point.

Present systems are typically ill adapted to treat large volume treatment zones or volumes in an efficient manner due to the small focal spot of the typical therapy applicators and other considerations. This makes it more difficult to justify and adopt thermal therapy from ultrasound sources in clinical practice and also increases the cost of the treatments. Thus, new methods are desired to make the treatments faster for example by enhancing the focal energy delivery. One such method to enhance ultrasound energy absorption is by microscopic gas bubbles. They can either be generated in the tissue or blood, or injected into the blood stream as preformed gas bubbles. These gas bubbles can also be used for tissue disintegration, for example for thrombolysis or other removal of tissue.

Similarly, low pressure amplitude ultrasound exposures can be used to disrupt the blood-brain barrier (“BBB”) when preformed microbubbles are injected in the blood stream. This is a field of increasing research with the potential to enable new treatments of brain and central nervous system (“CNS”) disorders. The BBB prevents passage of molecules from the vasculature into the brain tissue when the molecules are larger than around five hundred Daltons, thereby significantly reducing the efficacy of pharmaceuticals and other agents. FUS disruption of the BBB has been successfully used to deliver amyloid-beta antibodies, as described by J. F. Jordao, et al., in “Antibodies Targeted to the Brain with Image-Guided Focused Ultrasound Reduces Amyloid-Beta Plaque Load in the TgCRND8 Mouse Model of Alzheimer's Disease,” PLoS One 2010:5:e10549; large molecule chemotherapy agents, as described by M. Kinoshita, et al., in “Noninvasive Localized Delivery of Herceptin to the Mouse Brain by MRI-Guided Focused Ultrasound-Induced Blood-Brain Barrier Disruption,” Proc. Natl. Acad. Sci. USA, 2006; 103:11719-11723; and other large molecules of clinically relevant size, as described by J. J. Choi, et al., in “Molecules of Various Pharmacologically-Relevant Sizes Can Cross the Ultrasound-Induced Blood-Brain Barrier Opening In Vivo,” Ultrasound Med. Biol., 2010; 36:58-67.

It is therefore useful to be able to controllably generate microbubbles in the tissue or blood stream for enhanced delivery of ultrasound energy to a subject, or related therapeutic effects of acoustic cavitation, such that tissue ablation, thrombolysis, blood-brain barrier disruption or tissue diagnosis and similar purposes that can be achieved reliably without undesired injury to the subject.

### SUMMARY

Aspects hereof provide ways for treating and/or diagnosing diseased volumes in tissue using focused ultrasound transducer applicators and control of the same. In some aspects, generation and/or use of gas bodies by the ultrasonic waves is used and applied in a controlled manner to take effect of enhanced absorption of the higher frequency ultrasonic energy components at or near a focal spot.

Specifically, some present embodiments are directed to a method for applying acoustic energy to a target location, comprising exciting a first acoustic source to deliver a first acoustic field, having a first characteristic frequency, to a target location; exciting a second acoustic source, to deliver a second acoustic field, having a second characteristic frequency greater than said first characteristic frequency, to said target location; modulating said second acoustic field so as to cause a combined acoustic field from both the first and second acoustic fields at said target location to reach a threshold of cavitation during a portion of a cycle of said first acoustic field. The combined fields may also lead to pre-cavitation events, including nucleation of acoustically-excitable micro volumes of gas, vapor, bubbles, contrast agents or other cavities in or proximal to the target location.

In some aspects, the first lower frequency field may exceed or reach the threshold for causing cavitation or nucleation on its own, but the second field at the higher operating frequency can be applied at or slightly before or slightly after the acoustic minima of the first lower frequency field at the target location.

Other embodiments are directed to a system for delivering acoustic energy comprising a first acoustic source having a first characteristic response frequency; a second acoustic source having a second characteristic response frequency that is greater than said first characteristic response frequency; a controller including a modulating circuit that modulates a second driving signal to said second acoustic source, including gating said second driving signal relative to a phase of a first driving signal of said first acoustic source.

The present technique includes in some aspects controlling an amplitude of said first and second driving signals so that the acoustic (preferably ultrasonic) field at a target location has adequate pressure amplitude during its negative cycle or portion of its cycle to generate or excite microbubbles in the tissue or blood or microbubble precursors (for example superheated perfluorocarbon droplets) injected in the blood during the rarefactional phase of the wave. In this way, an ultrasonic dose of energy in said target location or volume experiences substantial interaction with the generated microbubbles. These generated microbubbles may provide therapeutic effects on the target tissue and/or may also be used for diagnostic purposes.

The method may be adapted to use variable frequency sonications (such as a chirp, where the frequency is either increased or decreased with a desired constant or variable rate) for each of the frequencies and/or use more than two frequencies and may also use unfocused ultrasound fields.

For microbubble or cavitation based treatments it is sometimes useful to localize the microbubbles to a zone at or near or in a targeted area such that the potential of unwanted damage from cavitation effects outside of the target tissue could be reduced or eliminated. One option is to generate the microbubbles at a focal zone with a high pressure amplitude ultrasound burst. This however, requires high pressure amplitudes at frequencies that provide precise focusing and may not be always achievable. The cavitation threshold reduces with decreasing frequency and can thus be achieved at lower frequencies. However, these lower frequencies do not provide precise localization of the therapy. Similarly, the use of low frequency ultrasound to induce cavitation effects has been shown to potentially cause blood vessel rupture and bleeding.

Another aspect includes the use of preformed perfluorocarbon droplets at or proximal to the target location that can be vaporized by ultrasound energy to cause and excite microbubbles under ultrasound exposure.

### BRIEF DESCRIPTION OF THE DRAWINGS

For a fuller understanding of the nature and advantages of the present concepts, reference is be made to the following detailed description of preferred embodiments and in connection with the accompanying drawings, in which:

FIG. 1 illustrates an exemplary focused ultrasound array;

FIG. 2 illustrates the use of an array to achieve an extended focal zone by additive contributions from several focal spots located at varying distances from the array;

FIG. 3 shows a graph of the cavitation threshold as a function of frequency for 1 sec exposure in vivo in thigh muscle;

FIG. 4 illustrates the use of an enhancer wave to carry the therapeutic signal waveforms and provide a composite wave containing more than one frequency component; (A) Ultrasound waveform produced by the first transducer; (B) ultrasound waveform produced by the second transducer of lower frequency; (C) combined application of the first and second transducers, where the ultrasound energy from the first transducer is delivered to the tissue while the second transducer of lower frequency is providing negative acoustic pressure;

FIG. 5 illustrates the use of two focused transducer sources to provide a composite waveform including an enhancer waveform and a modulated therapy signal wave at a common focus;

FIG. 6 illustrates a schematic exemplary system for providing a thermal therapy to a target volume using a plurality of driving signals and corresponding array element groups.

### DETAILED DESCRIPTION

Aspects of the present invention utilize a combination of two or more ultrasound frequencies in order to enhance focused ultrasound treatments or diagnostics such that the interaction of the two or more frequencies may enhance the therapeutic or diagnostic use of focused ultrasound systems and reduce the likelihood of deleterious effects occurring outside the area of therapeutic intervention or diagnostic investigation. The two frequencies may be different by a significant margin, for example where one frequency is one or more orders of magnitude lower than another frequency.

Embodiments hereof utilize a phased array applicator having at least one operating central response frequency, for example in the frequency range of 0.1-20 MHz. Alternatively, or in addition, geometrically focused transducers and arrays could be used. A resulting ultrasound beam is directed at the target tissue and sonications are applied using short, high power bursts (e.g., 1 to 100 kilo cycles) each of which is electronically or mechanically aimed at different locations. The sonications at a given location in space can be repeated multiple times to ensure that adequate temperature elevation has been achieved during thermal treatments to treat the given condition or disease. The interval between the bursts is used to control the overall rate of temperature elevation, for example to allow tissue temperature or other properties to be measured with adequate accuracy such that treatment control can be executed. Tissue temperature can be measured for example using magnetic resonance imaging (MRI) thermometry. Alternatively, the tissue stiffness change associated with tissue coagulation can be monitored using diagnostic ultrasound imaging to detect tissue stiffness changes and to infer the temperature or thermal dose.

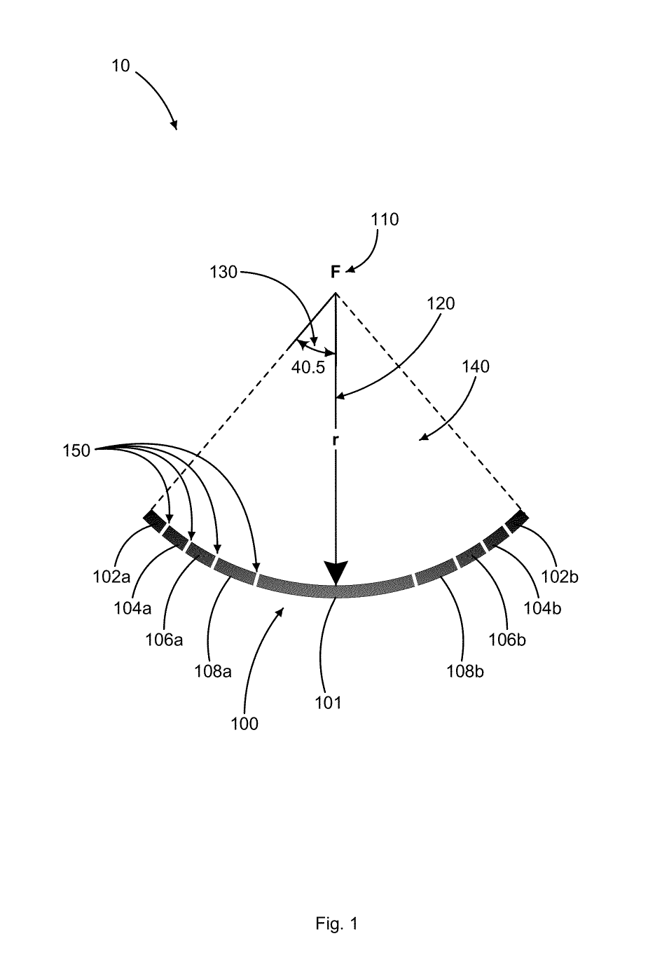

FIG. 1 illustrates a cross-section of an exemplary focused ultrasound array 10 that includes a plurality of array elements 100, e.g., piezoelectric transducer elements that geometrically form a focus at some point 110 (“F”) at a focal distance 120 (“r”) from the surface of the transducer.

Specifically, by driving transducer elements 100, ultrasonic sound waves emanate from the concave face of the transducer elements. The transducer 10 is cut using a plurality of cuts or kerfs 150 so that the array elements 100 are suitably sized. In some examples, roughly equal power is delivered to and from each of the elements of the array, causing the elements of the array to have approximately equal surface areas. The array shown in cross section in FIG. 1 has a center element 101, which is symmetrical about the axis of symmetry 120 of the transducer 10. An outermost transducer element 102a, 102b is annular in shape and conforms to the concave profile of the transducer, therefore 102a and 102b in FIG. 1 correspond to the same annular transducer in cross section. Similarly, annular array elements 104a-104b, 106a-106b and 108a-108b each have approximately the same surface areas as the center element 101. However, the present disclosure is not limited to the illustrative configuration above.

In total, the transducer 10 has an active area within which the ultrasonic waves propagate from the surface of the transducer elements to a focal spot 110 (“F”) a distance 120 (“r”) from the transducer's face and covering a conical half-angle 130. In its electrical design, the transducer elements 100 may have a common ground on one of their sides, but the elements 100 can be individually driven.

In some embodiments, the energy from all of the phased array elements are not focused to a single focus spot but rather are distributed, such that a first high pressure amplitude long focus along the direction of the wave propagation is formed in the target tissue.

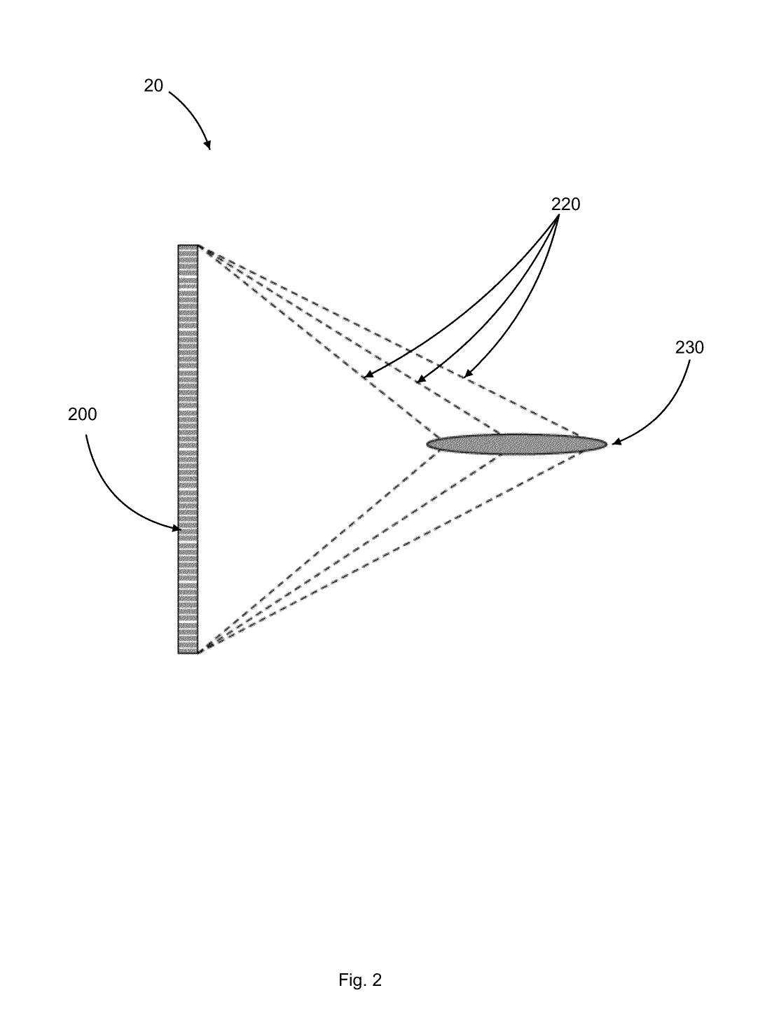

FIG. 2 illustrates a scenario 20 for treatment using a transducer array 200 having multiple transducer elements arranged along a line or plane in a “1.5 dimensional” or “2 dimensional” configuration. The phasing of the individual elements of array 200 can be accomplished to form a plurality of focal spots (or regions of high-intensity ultrasound resulting from the additive effect e.g., superposition) of the individual fields of the individual array elements. A plurality of propagation envelopes 220 can be formed by proper driving and configuration of array 200 so that a combined, elongated, focal zone 230 is formed by array 200.

The phasing may be carried out so that the multiple individual foci constituting elongated focal zone 230 are formed simultaneously, e.g., by driving separate groups of elements of array 200 at the same time, each group of elements providing one of such plurality of overlapping foci within elongated focal region 230. Alternatively, some or all elements of array 200 may be driven so as to form a first individual focus at a first distance from array 200, followed in time by a re-phasing or re-driving of said some or all elements of array 200 so as to form a second individual focus at a second distance from array 200 within elongated focal zone 230, and so on.

Note that in some embodiments the elements of the array 200 may be driven using a driving signal having a same (single) characteristic operating frequency to achieve the above result, but that in other embodiments the elements of array 200 are divided into more than one group and each group of elements is driven by a distinct driving signal having a correspondingly distinct center frequency, resonance frequency, mode, or simply, characteristic frequency of operation. In other words, in some embodiments, two or more subsets of elements of the array 200 are driven at two or more corresponding characteristic operating frequencies. It is to be understood that the present systems are limited by practical design and engineering considerations. For example, their size, geometry and operating frequencies are intended as described but small variations within accepted tolerances in the field are comprehended. So the operating frequencies are substantially as described herein, but may deviate, drift or have minor adjustments made to accommodate system operating conditions and so on. For example, a first group of elements may be driven at a higher characteristic frequency to form a series of first set of individual foci in extended focal zone 230 while a second group of elements may be driven at a lower characteristic frequency to form a second group of individual foci in extended focal zone 230. In some embodiments, the individual foci may be arranged substantially along the propagation direction(s) of the ultrasound waves.

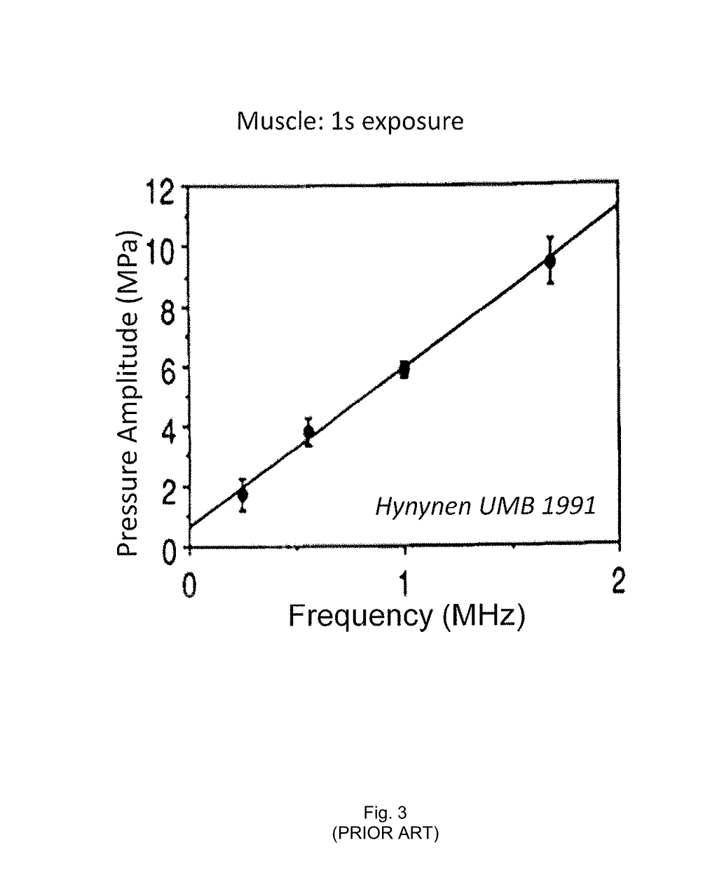

Aspects of the current disclosure utilize the phenomenon that the cavitation threshold reduces with decreasing frequency. FIG. 3 illustrates the dependence of the pressure threshold for cavitation on frequency for a 1 sec exposure in vivo in thigh muscle. Cavitation can thus be achieved with less pressure at lower frequencies.

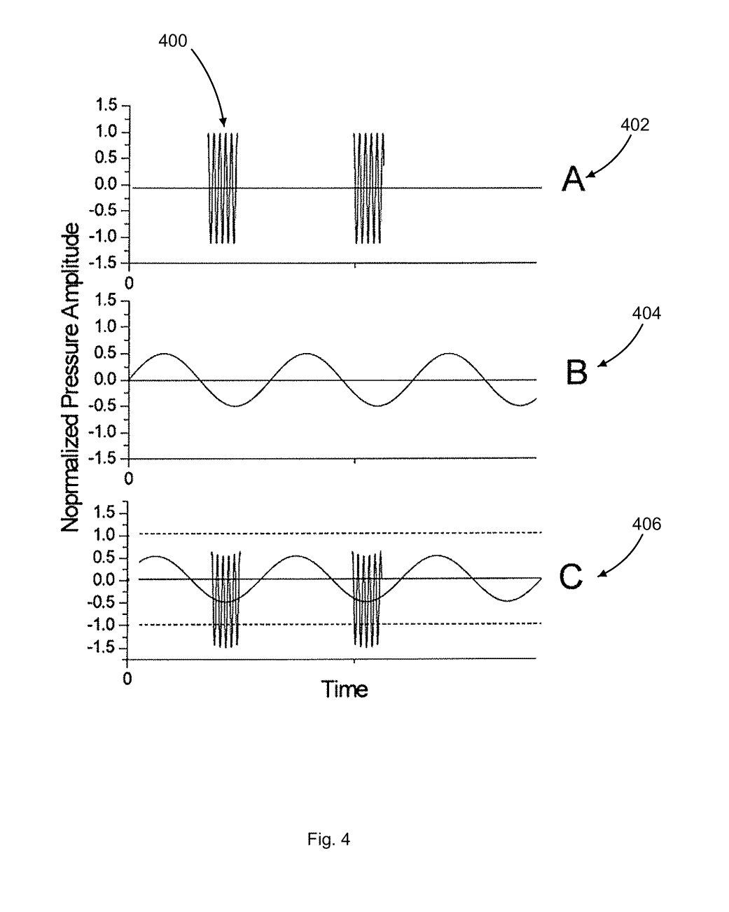

FIG. 4 illustrates a group of waveforms usable to obtain enhanced therapeutic effects from a thermal therapy applicator as described above. Here, multiple waveforms are generated and emitted by some or many elements of the transducer array. The multiple waveforms are provided to the target volume at substantially the same time so as to create a composite overall acoustic field comprising components from each of the multiple waveforms. In one embodiment, two waveforms, a high-frequency and a low-frequency waveform, are generated by the transducer and delivered to the target tissue for enhanced therapeutic effect. One or both waveforms may be gated, windowed, or temporally controlled so as to be ‘delivered’ in modulated bursts or packets. Moreover, one waveform at one central operating frequency may modulated in time (e.g., using amplitude modulation) relative to an amplitude of the second waveform at a target location. The central operating frequencies of the two waveforms may differ significantly (for example by one or more orders of magnitude) from one another. Therefore, the higher frequency waveform may be modulated or gated with respect to the acoustic amplitude of the lower frequency waveform.

Referring to FIG. 4, a first waveform 402 (A) consists of high frequency burst or sequence 400, which may be a high-frequency sinusoidal burst defined by a window so that within the window the high-frequency signal is present and outside the window the high-frequency signal is not present. This gating or modulating behavior can be achieved by multiplication or convolution of a base high-frequency signal with a gating or modulating envelope, which can be ON-OFF in nature or HIGH-LOW, +/−, square wave, saw tooth, sinusoidal or another modulating pattern. A second waveform 404 (B) consists of a significantly lower frequency signal than that of first waveform 402 e.g. being one or more orders of magnitude lower. The second waveform may be applied for a longer duration than the first high-frequency waveform to a common spatial location. Trace 406 (C) shows the combined first and second waveforms 402, 404, which would afford an additive or substantially additive result so that the transducer is seen to provide the combined or composite signal at the focus of the therapy device at the region of interest or target. Obviously it is irrelevant to the present disclosure which of the frequencies or groups of acoustic sources is referred to as the ‘first’ or the ‘second.’ A plurality of operating characteristic frequencies or groups of acoustic sources may be employed to achieve the present result in a variety of combinations as would become understood to those skilled in the art.

It can be appreciated that a plurality of waveforms (two or more) may be used to achieve the present purpose. For simplicity, we discuss a two-frequency system and method, but the present disclosure is not so limited.

The effect of applying the multi-frequency (e.g., two frequency) composite acoustic field would enhance the therapeutic effect. In part, this is because the first (high-frequency) signal 402 is modulated to arrive at the target location at a minimum in the low-frequency signal 404 cycle at that target location, thereby decreasing the peak positive pressures near the focus of the transducer and raising the peak negative (absolute) pressures at that location. The absolute increase in peak negative acoustic pressure is useful for enhancing cavitation effects at the target zone or focus of the transducer.

Those skilled in the art can appreciate that a DC offset (e.g., atmospheric or other static pressure field) can be applied to the present scenarios with no loss of generality, as the conditions for causing cavitation or nucleation of acoustically-excitable objects or voids in or around the target location can be achieved in such circumstances as well.

In operation, this may be accomplished by setting a first group of transducer elements to provide the lower frequency (e.g., 1 to 500 kHz) signal that is aimed and focused at the target zone, then a second group of transducer elements can provide the second high-frequency ultrasonic field (e.g., 100 kHz to 10 MHz) so that the high-frequency bursts arrive at the target zone at a time of minimum amplitude of the low-frequency signal at the target zone or focus.

In some embodiments, separate transducers may be used to generate each of the frequency components of the composite ultrasonic field. For example, a first focused transducer operating at 100 kHz may be combined with a second focused transducer operating at 1 MHz, both transducers being con-focused at about the same spatial focal region.

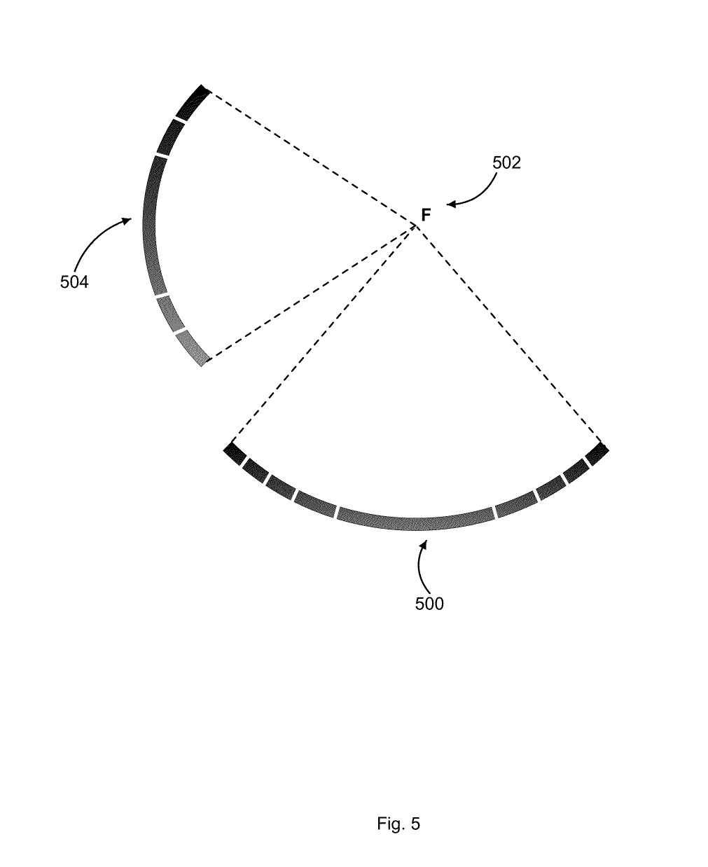

FIG. 5 illustrates a simplified example of such a dual-transducer, dual-frequency therapy configuration. A first (e.g., low-frequency) transducer 500 provides an ultrasonic field (e.g., 200 kHz center frequency) focused at a focus 502 (F). A second (e.g., high-frequency) transducer 504 is also focused at or near focal point 502 (F) and provides a modulated higher frequency (e.g., 2 MHz) burst signal timed so that it arrives when the lower frequency signal is at or near its peak negative amplitude at focus 502 (F). The composite or compound sound field may qualitatively look like the combined trace 406 of the previous figure.

As before, the elements of transducer 500 may be grouped into more than one group, each of which may be driven at distinct center frequencies and amplitudes. Alternatively, the groups may be driven at substantially the same center frequency. This notion of separately controllable elements and subgroups of elements can be carried to construct transducer arrays where each individual transducer element is in its own group so to speak, where each such individual element is separately controlled, even though the elements then operate in concert to achieve the resulting foci and therapeutic treatment result.

In some aspects, the energy deposition at the focus of a thermal therapy array is increased and the wave propagation beyond the focus can be reduced. This will translate in increased energy delivery with lower safety concerns, allowing faster and more economical treatments. In addition, the distortion of the ultrasound waves induced by the overlying tissues is minimized due to the long wavelengths that can be achieved. It is noted that the speed of sound in tissue is independent or generally less dependent on the ultrasound frequency, and thus, variations in the thickness of a fat layer in a patient (having a speed of sound lower than in other soft tissues) produces a frequency-independent time shift in the ultrasound wave front. The proportion of time shifts when compared with the wavelength are smaller with a lower frequency ultrasound applicators than with higher frequency applicators.

As mentioned earlier, in some embodiments, the therapy beam can be generated using a two dimensional phased array either with a full or limited beam steering capacity. Each of the elements of the phased array is driven by a radio frequency (RF) driving signal generated by a wave generator and amplified by an amplifier. The array elements may share some, all, or none of the signal generator and amplifier circuits among them.

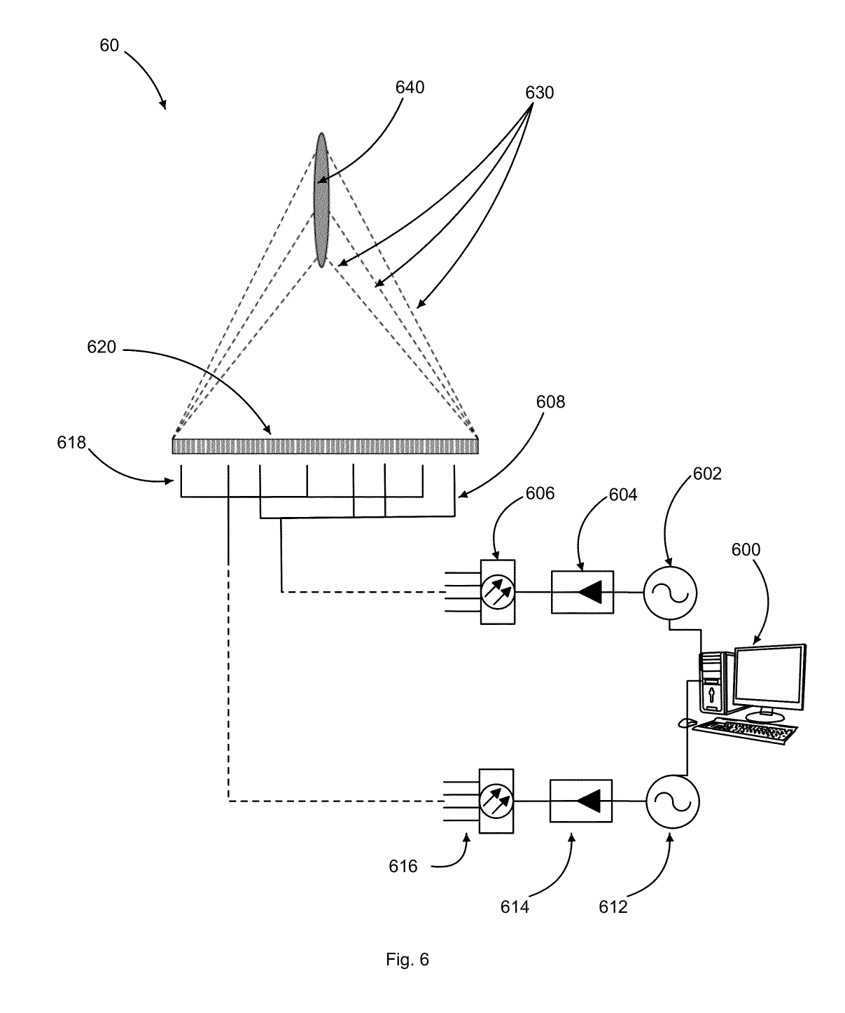

FIG. 6 illustrates schematically an exemplary system 60 for providing controlled thermal therapy from an ultrasonic array or arrays for treating a condition in the body of a patient. The condition could for example be a tumor, e.g., a cancerous group of cells in the patient's body, or other diseased tissue, including nervous, muscular, glandular, or circulatory tissue. A plurality of acoustical sources may be provided in one or more acoustical transducer arrays 620 to deliver acoustic energy to a target volume 640. In this example, an array 620 includes a plurality of sub-groups of elements 608 and 618, each respectively including a plurality of transducer elements to form acoustical foci at desired locations within the target location 640 in the patient's body.

A computer, work station, or processing apparatus 600 is configured and programmed to determine and deliver signals to a plurality of signal generators 602, 612. The signal generators 602, 612 may be incorporated in a single signal generating apparatus or be implemented as separate signal generating circuits. The signal generators 602 and 612 provide respective output signals at respective first and second characteristic operating frequencies. For example, the outputs from the signal generators may comprise cyclical (e.g., sinusoidal or saw tooth or square wave) signals having some general periodicity or respective central characteristic frequency. In some embodiments, the first signal generator 602 provides a first output signal having a first characteristic operating frequency f1, which may for example be in a range of 500 kHz to 10 MHz or some other useful therapeutic ultrasonic frequency. The second signal generating circuit 612 may provide an output having a lower characteristic periodicity or second characteristic operating frequency (e.g., in the range of 1 to 500 kHz), which can be used to enhance the therapeutic effect of the system.

Amplifiers 606, 616 amplify the driving signals from signal generators 604, 614 so that they deliver respective power levels to the array elements of respective first and second acoustical source arrays 608 and 618. That is, the frequency and amplitude of the electrical driving signals to the elements of the groups of transducers used may be determined and controlled by the system. Control of the phasing to each element can be used to form beams of ultrasonic energy to steer and control the spatial position of the resulting foci of the array elements operating in concert to deposit thermal energy at the desired target location 640 in the patient's body.

The first array 608 may be used to form a first of a plurality of foci at substantially the first characteristic operating frequency, and then the elements thereof may be steered or phased to form another focus and then another, resulting in an extended or elongated target location 640. The target location 640 will receive a controlled heating dose (or thermal dose) or be controlled and monitored to reach pre-determined temperature values so as to treat the condition at hand.

The second array or acoustical source 618 may be used to enhance the effectiveness of the treatment from the first array or acoustical source 608. The second acoustical source 618 delivers acoustic waves at substantially the second characteristic operating frequency at or near target location 640. The system is controlled so that the first acoustical source 608 provides a pulse, group, or packet of waves at target location 640 substantially concurrent with or slightly delayed from the second acoustical source 618 delivering a peak negative acoustic pressure at the target location 640. In this way, as discussed earlier, the system delivers an effective dose of ultrasound energy to the target 640 when there is a maximized likelihood and extent of gas bubble formation at the target location 640.

Collectively, and specifically through application of appropriate control and ultrasonic energy levels and frequencies, increased cavitation and thermal energy deposition takes place in and proximal to the focal areas described above.

The amplitude, phase, and frequency of the waves emitted by each array element or group of elements may be controlled by a general computer (e.g., PC or workstation) running machine-readable software, or by a special purpose processor executing instructions thereon. For example the methods can be used to generate and amplify the driving signals and resulting ultrasound waves. The RF signals from the multi-channel driver may be connected to each of the phased array elements for example by way of a coaxial RF line.

The ultrasound waves generated by the phased array are coupled to the target tissue for example through direct contact or through a liquid or solid coupling layer, acoustic gel, or medium. The properties of the coupling medium may be chosen to optimally shape the waveform for use in the therapy application, for example as given above.

The present invention should not be considered limited to the particular embodiments described above, but rather should be understood to cover all aspects of the invention as fairly set out in the present disclosure. Various modifications, equivalent processes, as well as numerous structures to which the present invention may be applicable, will be readily apparent to those skilled in the art to which the present invention is directed upon review of the present disclosure.

What is claimed is:

1. A method for applying acoustic energy to a target location, comprising:
exciting a first acoustic source to deliver a first acoustic field, having a first characteristic frequency, to a target location;
exciting a second acoustic source, to deliver a second acoustic field, having a second characteristic frequency greater than said first characteristic frequency, to said target location;
modulating said second acoustic field so as to cause a combined acoustic field from both the first and second acoustic fields at said target location to reach a threshold of cavitation or gas body nucleation during a portion of a cycle of said first acoustic field.

2. The method of claim 1, exciting said first acoustic source comprising driving one or more transducers with a first driving signal having said first characteristic frequency.

3. The method of claim 1, exciting said second acoustic source comprising driving one or more transducers with a second driving signal having said second characteristic frequency.

4. The method of claim 1, exciting said first and second acoustic sources comprising exciting respective first and second transducers or groups of transducers in a single multi-transducer assembly or array.

5. The method of claim 1, exciting said first acoustic source comprising exciting a first transducer or group of transducers in a first acoustic assembly or array and exciting said second acoustic source comprising exciting a second transducer or group of transducers in a second acoustic assembly or array different from the first assembly or array.

6. The method of claim 1, exciting said first acoustic source comprising driving said first acoustic source with a first periodic driving signal having said first characteristic frequency.

7. The method of claim 1, exciting said second acoustic source comprising driving said second acoustic source with a second periodic driving signal having said second characteristic frequency that is greater than said first characteristic frequency.

8. The method of claim 7, said second characteristic frequency being at least an order or magnitude greater than said first characteristic frequency.

9. The method of claim 1, said modulating comprising modulating an amplitude of said second acoustic field relative to a phase of said first acoustic field.

10. The method of claim 9, modulating said amplitude comprising gating said second acoustic field so as to apply the second acoustic field during gated time windows.

11. The method of claim 10, said gating timed so as to deliver bursts of said second acoustic field at said target location during times at which an acoustic pressure of said first acoustic field at the target location is at relative negative minima.

12. The method of claim 1, further comprising focusing at least one of said first and second acoustic sources onto a focal region including said target location.

13. The method of claim 12, comprising geometrically focusing at least one of said first and second acoustic sources onto said focal region.

14. The method of claim 12, comprising controlling a phase of respective driving signals applied to at least one of said first and second acoustic sources where said at least one of said first and second acoustic sources comprise discrete elements of a phased array.

15. The method of claim 1, modulating said second acoustic field comprising modulating said second acoustic field relative to an acoustic pressure of said first acoustic field at said target location.

16. The method of claim 1, modulating said second acoustic field comprising modulating said second acoustic field relative to a phase of an excitation of said first acoustic source.

17. The method of claim 1, further comprising generating cavitation gas bodies at or proximal to said target location with said combined acoustic field.

18. The method of claim 1, further comprising exciting pre-existing gas bodies present at or proximal to said target location with said combined acoustic field.

19. The method of claim 1, further comprising exciting pre-existing gas body precursors at or proximal to said target location with said combined acoustic field.

20. The method of claim 1, said first acoustic field reaching a sufficient negative acoustic pressure at its minima so as to reach said threshold at said target location even in the absence of said second acoustic field.

21. A system for delivering acoustic energy comprising:
a first acoustic source having a first characteristic response frequency;
a second acoustic source having a second characteristic response frequency that is greater than said first characteristic response frequency;
a controller including a modulating circuit that modulates a second driving signal to said second acoustic source, including gating said second driving signal relative to a phase of a first driving signal of said first acoustic source.

22. The system of claim 21, at least one of said first and second acoustic sources comprising a phased array of discrete transducers.

23. The system of claim 21, at least one of said first and second acoustic sources comprising a geometrically focused acoustic source.

24. The system of claim 21, said second characteristic response frequency being at least an order of magnitude greater than said first characteristic response frequency.
